(19) United States
(12) Patent Application Publication
(10) Pub. No.: US 2004/0176436 A1
(43) Pub. Date: Sep. 9, 2004

(54) Tryptophanyl-esters and their N-acyl derivatives for preventing and treating diseases caused or aggravated by oxidation processes

(76) Inventor: Christian Behl, Munchen (DE)

Correspondence Address: FISH & NEAVE, 1251 AVENUE OF THE AMERICAS, 50TH FLOOR, NEW YORK, NY, 10020-1105, US

(21) Appl. No.: 10/788,608

(22) Filed: Feb. 27, 2004

Related U.S. Application Data
(62) Division of application No. 09/810,152, filed on Mar. 16, 2001, now abandoned.
Application No. 09/810,152 is a continuation of application No. PCT/EP99/06819, filed on Sep. 15, 1999.

(30) Foreign Application Priority Data
Sep. 16, 1998 (DE) 198 42 416.7

(51) Int. Cl. A61K 31/405

(52) U.S. Cl. 514/419

(57) ABSTRACT

Use of a tryptophanyl-ester or an N-acyl derivative of a tryptophanyl-ester for the prophylaxis and therapy of oxidative pathologic processes in degenerative diseases and/or cancer diseases. Thereby, the tryptophanyl-ester or its N-acyl derivative is preferred to be tryptophanoctyl, N-oleoyl-tryptophanethyl-ester or N-dodecanoyl-tryptophanethyl-ester.

Furthermore the tryptophanyl-esters and their N-acyl derivatives are preferred to be used for the treatment and/or prophylaxis of neurodegenerative diseases, cataracts, neoplastic diseases and/or cardiovascular diseases. In particular, the invention relates to the use of the tryptophanyl-esters and their N-acyl derivatives for the treatment and/or prophylaxis of Alzheimer's disease, Parkinson's disease, apoplectic fit, amyotrophic lateral sclerosis, cancers, arteriosclerosis and/or myocardial infarction.

In addition, the invention relates to a pharmaceutical composition comprising a tryptophanyl-ester or an N-acyl derivative of a tryptophanyl-ester.

The invention relates to the use of tryptophanyl-esters and their N-acyl derivatives for preventing and treating diseases which are caused by oxidative processes, i.e. so-called oxidative stress, or which are accompanied by unintended oxidation of cellular molecules. Such diseases known comprise both degenerative diseases like Alzheimer's disease, Parkinson's disease, amyotrophic lateral sclerosis (ALS), apoplectic fit, myocardial infarction, atherosclerosis, cataracts, and various kinds of cancer like breast carcinoma, melanoma, brain tumours.

It is known that oxidative processes play a central role with regard to both the pathogenesis of various degenerative diseases and the origin of uncontrolled cell growth of various kinds of cancer. Oxidants are usual by-products of the cellular metabolism, they can, however, also damage proteins, fatty acids (lipids) and deoxyribonucleic acids (DNA) if they are not efficiently changed into harmless compounds in time. Oxidative damage to central functional units of the cells trigger the formation of cancer (Ames, Science, vol. 221, pp. 1256-1264, 1983), cause cardiovascular diseases (e.g. arteriosclerosis, myocardial infarction, cf. Parthasarathy, Annu. Rev. Med., vol. 43, pp. 219-225, 1992), lead to immunodeficiency and, above all, to disorders as regards cerebral functions, e.g. memory defects, circulatory disorders (Coyle and Puttfarcken, Science, vol. 262, pp. 689-695, 1993), to neurodegenerative diseases and death of the cerebral cells (e.g. Alzheimer's disease, Parkinson's disease, apoplectic fit, ALS) (Coyle and Puttfarcken, Science, vol. 262, pp. 689-695, 1993; Beal, Ann. Neurol., vol. 38, pp. 357-366, 1995) as well as to various degenerative geriatric disorders (for an overview see Ames, Proc. Natl. Acad. Sci. USA, vol. 90, pp. 7915-7922, 1993). The oxidation of macromolecules also plays a crucial role in the formation of ateriosclerosis. In this case, the so-called low-density lipoprotein (LDL) is found in the deposits of arteriosclerotic vascoconstrictions. Oxidated LDL has increased aggregation features. With regard to a prevention and/or therapy of arteriosclerosis, it is the objective to prevent the oxidation of LDL with antioxidants (Parthasarathy et al., Annu. Rev. Med., vol. 43, pp. 219-225, 1992).

Oxidative stress is also considered to initiate the formation of cataracts since an extensive oxidation of proteins of the visual lens is, amongst others, accompanied by human cataracts. Furthermore, the peroxide level in cataract tissue is increased (Spector, FASEB J., vol. 9, pp. 1173-1182,1995). The accumulation of peroxides is a general mechanism which mediates oxidative stress (Ames, Science, vol. 221, pp. 1256-1264, 1983; Behl, Progress in Neurobiology, vol. 57, pp. 301-323, 1999): Hence, oxidative stress is one of the most important reasons for the origin of the most varied diseases. Although there are, in the organism and on cell level, endogenbus antioxidation systems, they are insufficient by far in an oxidative environment that is due to age or pathologically increased, e.g. during and after an apoplectic fit, myocardial infarction, ALS or with Alzheimer's disease and lead to the oxidative changes that are characteristic to said diseases.

In general, the body has to be supplied with natural antioxidants (e.g. vitamins C and E) in food (e.g. in fruit and vegetables) or they have to synthesized by the body itself (e.g. glutathione) in order to protect the cells against oxidative damage and oxidative diseases in the long run. Said natural antioxidation, however, is totally insufficient with pathological events or during oxidative events associated with age. Thus, it is necessary to provide novel, in particular, more effective antioxidants having a simple chemical structure, which reach the cellular target regions of oxidative processes (e.g. cellular membrane systems) at a high concentration due to their good solubility in fat (high lipophilia).

Surprisingly, such novel antioxidants having a high lipophilia were found for the first time by means of an experimental paradigm as to the oxidative cell death using the tryptophanyl-esters and their N-acyl derivatives presented herein.

Although it has been known for a long time that oxidative processes damage the vital structures of the cell and cause pathological changes, due to a lack of suitable potent antioxidants, there have so far only been therapies that are not very effective and currently extremely unsatisfactory and that could be used purposefully for preventing said pathological processes (Behl, Progress in Neurobiology, vol. 57, pp. 301-323, 1999).

Apart from vitamins E and C, only polycyclic phenol compounds are known as potential preventive and therapeutic antoxidants. Starting from the female sexual hormone estrogen, such polycyclic phenol compounds and lipophilic aromatic alcohol compounds have been identified and suggested as structures that are antioxidatively and neuroprotectively effective (Behl et al., Biochem. Biophys. Res. Commun., vol. 216, pp. 473-482 1995; Behl et al., Molecular Pharmacology, vol. 51, pp. 535-541, 1997; Moosmann et al., FEBS Letters, vol. 413, pp. 467-472, 1997).

However, decisive disadvantages have to be taken into consideration when polycyclic phenol compounds and estrogen derivatives are used. Such decisive disadvantages of the potential antioxidant estrogen, various estrogen derivatives and polycyclic phenol compounds which prevent a targeted therapeutic application include:

1 . The low efficiency, i.e. for a use in therapy it would be necessary to apply comparatively high concentrations of the substances. In the experimental approach, EC₅₀ values in a micromolar range are necessary to achieve a 50% cell protection against oxidation (Goodmann et al., J. Neurochem., vol. 66, pp. 1836-1844, 1996; Behl et al., Molecular Pharmacology, vol. 51, pp. 535-541, 1997; Moosmann et al., FEBS Letters, vol. 131, pp. 467-472, 1997).

2. The polycyclic phenol compounds have a high affinity to the estrogen receptor, thus leading to completely undesired feminising side-effects (Katzenellenbogen, Environmental Health Perspectives, vol. 103, pp. 99-101, 1995; Miksicek, Proc. Society for Exp. Biol. & Med., vol. 208, pp. 44-50, 1995; Ojasoo et al., Steroids, vol. 60, pp. 458469, 1995; Cook et al., Regulatory Toxicology & Pharmacology, vol. 26, pp. 60-68, 1997; Richardmeier et al., General & Comparative Endocrinology, vol. 100, pp. 314-326, 1995).

3. The activation of estrogen receptors by estrogens or estrogen derivatives and phenols is considered to be the origin of the formation or the aggravation of various kinds of breast cancer (Biswas, Molecular Medicine, vol. 4, pp. 454-467, 1998; White and Parker, Endocrine-related Cancer, vol. 5, pp. 1-14, 1998; Khan, J. Natl. Cancer Inst., vol. 90, pp. 37-42, 1998; Santodonato, Chemosphere, vol. 34, pp. 835-848, 1997).

4. A potential use of said substances has been described with limitation to neurodegenerative processes (e.g. Simpkins and Gordon, WO 97/03661).

5. In vivo, phenolic compounds are often in equilibrium with derived forms (e.g. glycosylated and acetylated derivatives) so that it is not possible to achieve high concentrations a the target site, e.g. in the brain (Forth, Henschler, Rummel, Starke: Pharmakologie und Toxikologie-Lehrbuch, BI-Wissenschaftsverlag, 1992; Gonzalez, Med. Hypotheses, vol. 32, pp. 107-110, 1990; Martucci and Fishman, Pharmacol. Therapy, vol. 57, pp. 237-257, 1993; Zhu and Conney, Carciogenesis, vol. 19, pp. 1-27, 1998).

6. The aforementioned compounds are capable of redox-cycling. Redox-cycling is a pro-oxidative effect which is said to be responsible for pathological processes in the Parkinson's disease (Ebadi et al., Progress in Neurobiology, vol. 48, pp. 1-19, 1996) and which, in general, counteracts with the antioxidative concept, i.e. metabolites of the phenolic antioxidants are pro-oxidants (Ames, Science, vol. 221, pp. 1256-1264, 1983; Thompson, Chem. Res. Toxicol., vol. 8, pp. 55-60, 1995; Gut, Environm. Health Perspect., vol. 104, suppl. 6, pp. 1211-1218, 1996).

7. Various phenols also behave in a pro-oxidative way under certain conditions (phenol/oxygen partial-pressure ratio; presence of copper or other heavy metal ions) (Yamashita, Chem. Res. Toxicol., vol. 11, pp.-855-862, 1998). Such conditions are likely to be also present in vivo, which additionally renders the use of phenols as antioxidants questionable.

Such decisive disadvantages make the use of said substances for a successful prevention and therapy very hard or may also be contraindicated (e.g. for a use in a male organism). Thus, the technical problem underlying the present invention was to provide a pharmaceutical composition and its use which may be used purposefully for preventing and, in particular, for treating oxidative pathologic changes without triggering the aforementioned side-effects.

Therefore, the use of tryptophanyl-esters and their N-acyl derivatives according to claims 1 to 11, a pharmaceutical preparation according to claim 12 and therapeutic methods according to claim 13 are subject matters of the invention.

Lipophilic tryptophanyl-esters and their N-acyl derivatives have an antioxidative effect in significantly lower EC₅₀ concentrations (in a medium nanomolar range, i.e. up to 100-fold better than polycyclic phenol compounds), which is pharmacologically desirable and makes it possible to achieve suitable effective concentrations of the substances at the target site (e.g. brain) from point of view of pharmacology. In particular, said lipophilic tryptophanyl-esters and their N-acyl derivatives do not have any affinity to estrogen receptors and, thus, do not have any estrogen receptor activating effect.

It was surprisingly found that the aforementioned tryptophanyl-esters and their N-acyl derivatives have a distinct antioxidative and cytoprotective effect against oxidative pathologic processes relevant with regard to said diseases.

In almost all oxidative processes in the cell electrophilic species with unpaired electrons, i.e. free radicals are involved in at least one decisive reaction step. Since such reactions are not kintetically inhibited (depending on the kind of electrophil, triplet oxygen reactions are kinetically inhibited to a great extent, as a consequence, triplet oxygen (oxygen in a normal state) causes no or only little oxidative strss; superoxide radical anion reactions are not kinetically inhibited, they cause strong oxidative stress. Since these reactions have high velocity constants, small well-diffusing molecules, which, at the respective site, interfere with the reaction mechanism of the possibly damaging electrophils and provide an alternative reaction path, are necessary to prevent such oxidative processes. In the case of the lipophilic tryptophanyl-esters and their N-acyl derivatives, said alternative reaction path could be the energetically preferred and kinetically fast provision of a hydrogen radical (H*) which reacts with the electrophil (E*) and, thus, leads to a chemically inert valence with the coupled pair of electrons (E-H). Although a new antioxidant radical (A*) would be formed, said radical is energetically so inert that it is not able to react with the usual cellular nucleophils due to the aromatic system in the direct neighbourhood and the nitrogen atom that is rich in electrons. Thus, its life time is long enough to achieve, by means of diffusion, one of the special cell-endogenous one-electron reduction systems, such as reduced nicotine adenine dinulceotide (NADH) and to be recycled by said system. This is one way of explaining the antioxidative effect of the trytophanyl-esters and their N-acyl derivatives which should not be used as a limitation.

Apart from avoiding estrogen-like side-effects and metabolic problems (see above, items 1-7), the decisive advantage of using tryptophanyl-esters and their N-acyl derivatives is that there is a molecule that can only be formed if nitrogen is used, said molecule having at the same time a labile hydrogen atom and the hydrogen carrier (nitrogen) of said molecule being stabilised by two aromatic systems (trivalence of t h e nitrogen). Using oxygen as hydrogen carrier, as is done in many phenolic antioxidants such as vitamin E and estrogen, only permits one aromatic system apart from the labile hydrogen atom. Thus, the superiority of the lipophilic tryptophanyl-esters and their N-acyl derivatives is due to the basic chemical nature and is expressed in the effect that surprisingly is 100 times more effective compared to the phenols.

Tryptophanyl-esters and their N-acyl derivatives having the following formula (I)

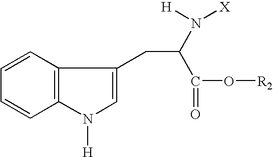

wherein X is C(O)R1 or H and R1 and R1 and R2 are, independently of each other, saturated or unsaturated C₂-C₁₈ carbon hydrogen residues and the tryptophanyl structure may be present in D- or L-configuration, have a distinct cytoprotecive and antioxidative effect against oxidative and degenerative pathologic processes. In this case, tryptophanoctyl-esters, N-oleoyl-tryptophanethyl-esters and N-dodecanoyl-tryptophanethyl-esters are particularly preferred embodiments.

All substances which may be administered in a pro-form and which, in the body, may be metabolised to one of the above-defined effective structures are included.

The compounds used according to the invention are generally known or can be produced according to a method which is known to the person skilled in the art (Beilste ins Handbuch der Organischen Chemie, main and supplementary works, years 1909-1979).

The present compounds can be administered by different routes in order to obtain the desired effect. The compounds can be administered to the patient treated alone or in the form of pharmaceutical preparations either orally or parentally, for example subcutaneously, intravenously, intramuscularly, or intracerebrally. They can also be administered by inhalation or by suppositories. They can, however, also be administered differently, e.g. transdermally, provided that the required doses can be obtained. The quantity of the administered compound can vary and it can be any quantity which is useful for the prophylaxis and/or therapy of oxidative pathologic processes and/or cancer. Depending on the patient, the gravity of the disease and the kind of administration, the administered quantity of the compound can vary extensively whereby approximately 0.1 mg/kg to approximately 10 mg/kg, usually from 1 to 5 mg/kg weight of the patient per dose are provided. Uniform doses of these compounds can, for example, contain 10 mg to 100 mg, usually 50 to 500 mg and preferably 250 to 500 mg of the compound and can, for example, be administered one to four times a day.

In this context, the term “uniform doses” means a form of a single or multiple dose containing a quantity of a substance in admixture or otherwise in combination with a diluting agent or the carrier, whereby the quantity is such that usually one ore more predetermined units are required for a single therapeutic administration. With a multiple doses form, like for example solutions or notched tablets, the predetermined unit is a fraction of the multiple doses form, like a 5 ml quantity (tea spoon) of a solution or a half or a quarter of a notched tablet.

Individual formulations of the invention are produced according to a method which is usually generally known in pharmacy and usually comprise at least one active substance according to the invention in admixture or otherwise in combination with a pharmaceutically acceptable carrier or diluting agent. For the production of this formulation the substance is usually mixed with a carrier or diluted with a diluting agent and then filled or encapsulated into a capsule, a gelatine capsule, a bag or another container. The carrier or diluting agent can be solid, half-solid or fluid material which can be used as carrier, excipient or medium for the substance. Suitable carriers or diluting agents are generally known. A description of the production of such formulations can be found in Remington's Pharmaceutical Sciences, Mack Publishing Company, Easton, Pa.

The compounds used according to the invention can preferably be used in therapy or prophylaxis of Alzheimer's disease or amyotrophic lateral sclerosis alone or in combination with other therapeutical compositions which have a different mode of action. Such pharmaceutical compositions include, for example, for the therapy and/or prophylaxis of Alzheimer's disease, ciliary neurotropic factor (CNTF) and aniracetam, buflomedil, choline, co-dergocrine, cyclandelate, desferrioxamine, eptastigmine, fampridine, galantamine; isoxsuprine, lecithin, linopiridine, metriphonate, naftidrofuryl, nicergoline, nicotine, nimodipine, oxiracetam, physostigmine, pilocarpine, piracetam, pramiracetam, propentofylline, pyritinol, RS-86, selegiline, suronacrine, tacrine, velnacrine, and for the therapy of amyotrophic lacteral sclerosis, somatomedines, protirelin, immunoglobulines and immunosupressiva.

The above-mentioned compounds can be formulated into pharmaceutical preparations according to a method known to the person skilled in the art, optionally with a conventional carrier material.

The above-mentioned compounds as pharmaceutical preparations can be administered orally, rectally, intravenously, intramuscularly, intracerebrally, parenterally or by inhalation. They can also be administered in a different way, for example transdermally, as long as the necessary dosage can be achieved.

The Figures show:

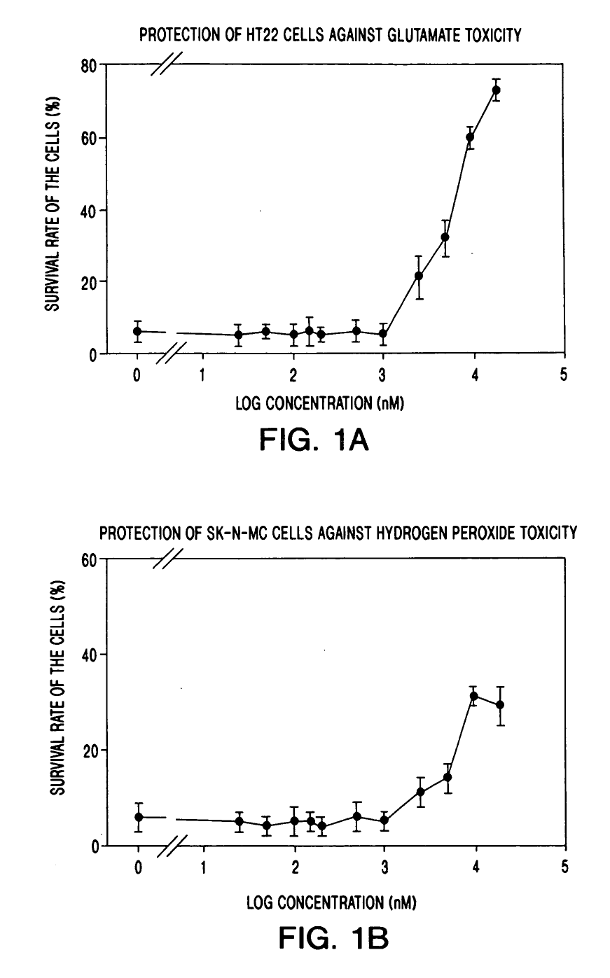

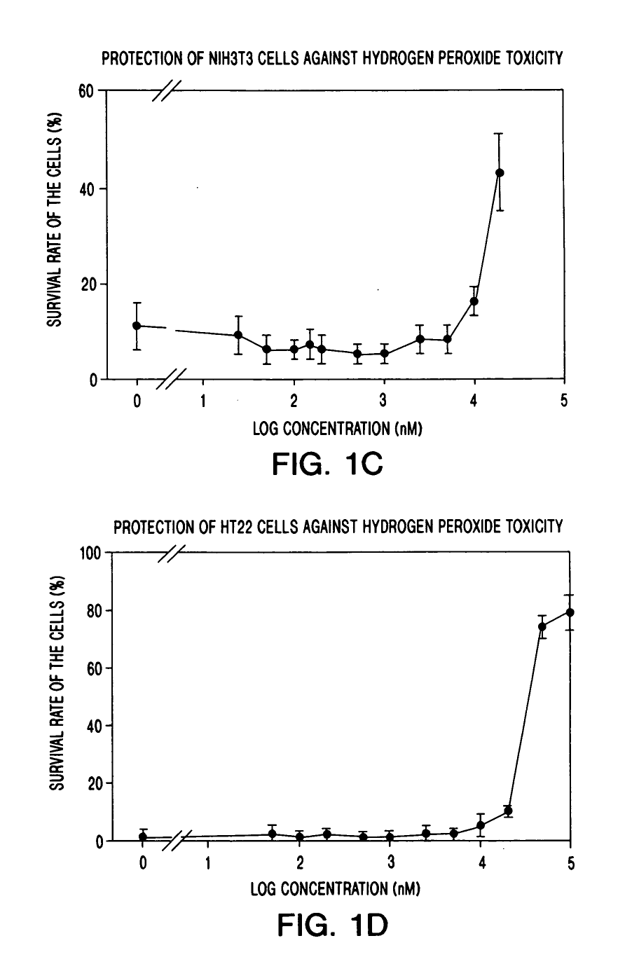

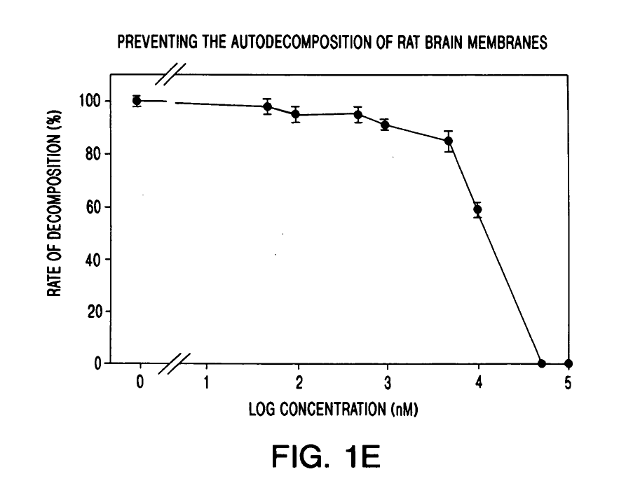

FIG. 1: Illustration of effects characteristic for tryptophan-octyl-ester

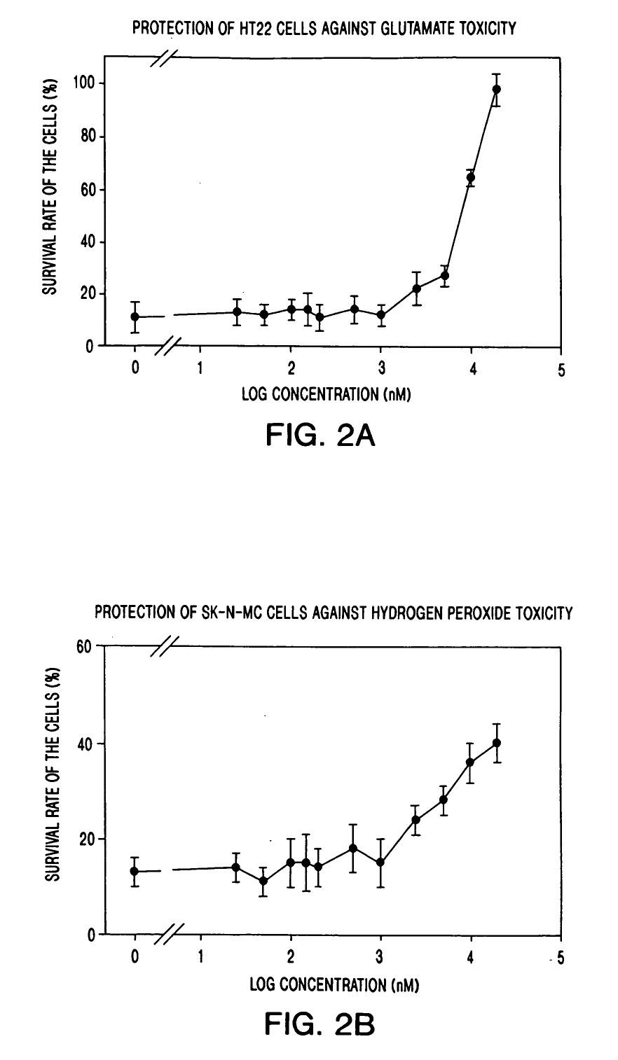

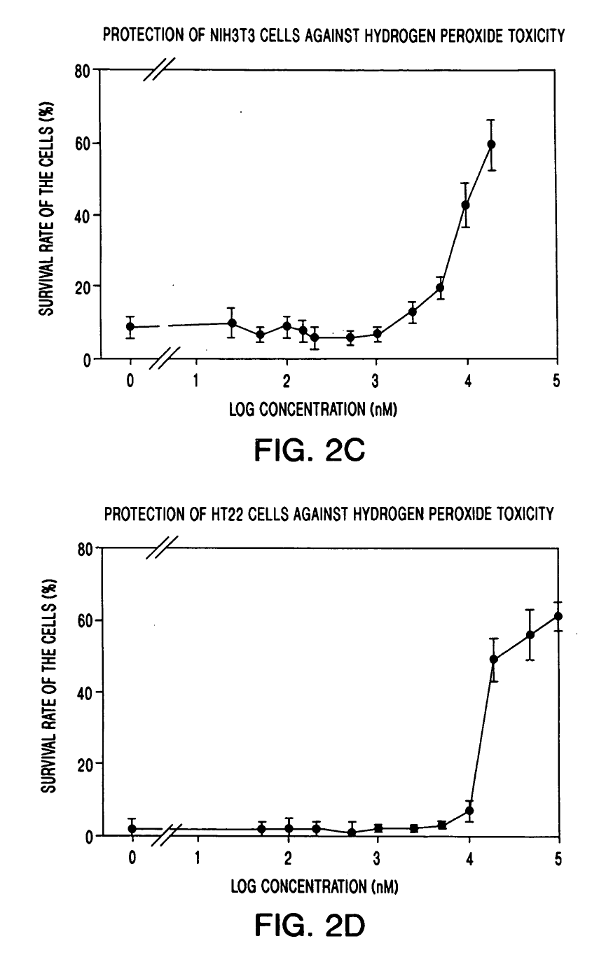

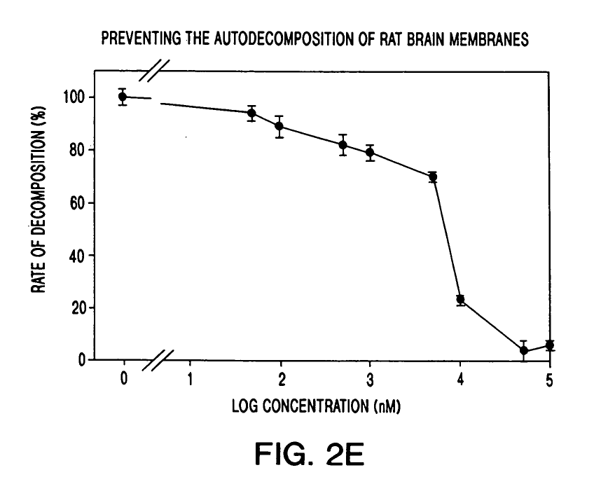

FIG. 2: Illustration of effects characteristic for N-oleoyl-tryptophan-ethyl-ester

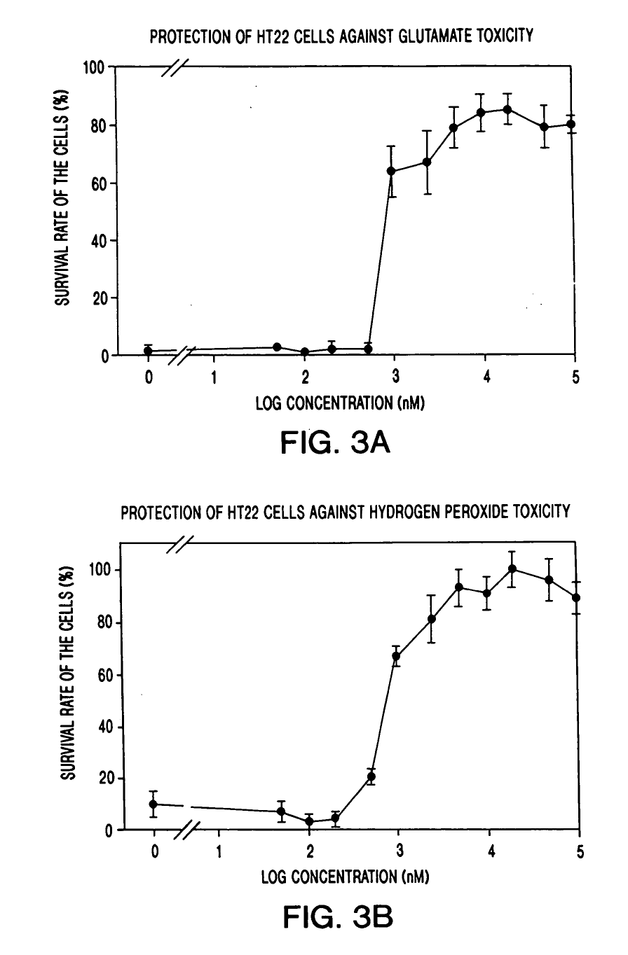

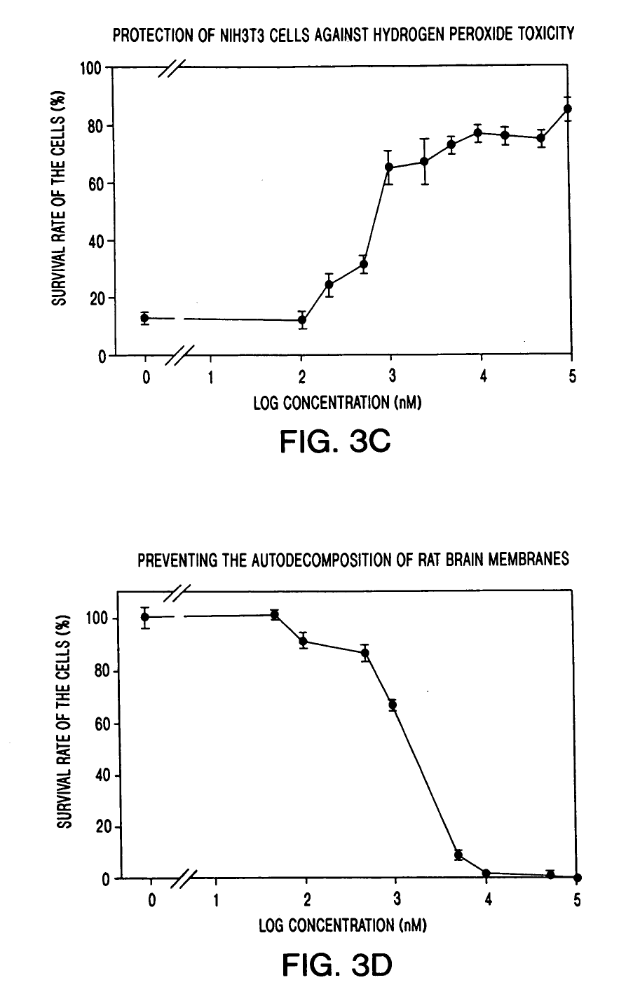

FIG. 3: Illustration of effects characteristic for N-dodecanoyl-tryptophan-ethyl-ester

The efficiency of the use of tryptophanyl-esters and its N-acyl derivatives according to the invention is confirmed by the following tests.

### EXAMPLE 1

As experimental system, nerve cells (clonal hippocampal cells from mouse, so-called HT22-cells; Morimoto and Koshland, Neuron, Vol. 5, pp. 875-880, 1990; and clonal human neuroblastoma cells, so-called SK-N-MC-cells; ATCC#HTB10) and murine fibroblast cells, so-called NIH3T3-cells (ATCC#CRL 1658), were put under oxidative stress in the cell culture. Oxidative stress was caused by acknowledged oxidants often used in the literature which lead to cell death due to peroxidation of cellular macromolecules within a short period of time: in the case of nerve cells, for example, by means of the excitatory amino acid glutamate and hydrogen peroxide, and in the case of the fibroblast, by means of hydrogen peroxide only. The number of surviving cells was determined by various standardised tests, particularly by measurement of the cellular mitochondrial activity. The results thereof were confirmed by subsequent microscopic determination of the number of cells.

Cells (e.g. HT22, SK-N-MC, NIH3T3) are plated into standard culture medium (DMEM supplemented with 10% foetal bovine serum) in culture dishes. The cells are left there overnight to guarantee the quantitative settling of the cells on the culture surface. The cell density during plating was 10% for HT22, 25% for SK-N-MC and 15% for NIH 3T3.

Then, the potential neuro-protective substances are added in various end concentrations (duration of incubation between 2 and 4 hours). The cell cultures are then incubated with the above-defined oxidative stressors, for example with glutamate or with hydrogen peroxide, in different concentrations each. The concentration of the oxidative stressors is chosen in such a way that the vitality of the control cells which have not been pre-incubated with the protective substances is lower than 15%, i.e. 3 mM (HT22) for glutamate and 160 μM (SK-N-MC) or 200 μM (NIH 3T3) for hydrogen peroxide. The incubation is carried out for approximately 20 hours before 3-(4,5-dimethylthiazol-2-yl)-2,5-diphenyl-tetrazolium bromide (MTT) (end concentration 0.5 mg/ml, Sigma) is added to the cells to determine the mitochondrial activity of the culture cells. MTT is turned into a blue formazan colouring agent by mitochondrial enzymes of the cell. The colour reaction is carried out for 4 hours. The reaction is then terminated by adding a detergent solution (10% SDS/50% DMF, pH 4.8) and read in a photometer at a wave length of 570 nm.

The protocol described herein is a version of the protocol originally published (Hansen, J. Immunol. Meth., Vol. 119, pp. 203-210, 1989) modified for cultivated nerve cells (cf. Behl, Biochem. Biophys. Res. Comm., Vol. 186, pp. 944-950, 1992).

In another survival test, the colouring agent trypan blue is added to the cells after completion of the toxin reaction in order to confirm the MTT data. Trypan blue can penetrate damaged membranes of dead cells only. The dead cells turn blue and can be counted under the microscope. The number of living, non-coloured cells and of dead cells is determined exactly. These experimental systems for the detection of anti-oxidative cyto-protective substances can be used in a highly reproducible manner.

The MTT and the cell number test are carried out according to standard methods (Behl, Cell, Vol. 77, pp. 817-827, 1994).

Protocol of the Brain Membrane Disintegration Experiment:

Native rat brain membranes were oxidated in a reaction catalysed by small doses of ascorbate (50 μM) (for details,see Moosman and Behl, PNAS 96, pp. 8867-8872, 1999). This reaction involves the occurence of primary oxidation products excited electronically which transform to their original state by the release of photons. By measuring the emitted single photons, the decomposition process of the brain membranes can be observed in real time and may be integrated chronologically. The measurement was carried out with a Beckman single photon counting scintillation counter six hours after adding ascorbate.

The results show that the substances can also protect pre-damaged brain membranes from further destruction, independently from the presence of endogenous protection mechanisms such as activated immune cells.

### EXAMPLE 2

Tryptrophan-octylester was tested in the concentrations 25 nM to 20 mM, as described in Example 1, with HT22 cells, SK-N-MC cells, NIH3T3 cells and rat brain membranes. The result is shown in FIG. 1.

### EXAMPLE 3

N-oleoyl-tryptophan-ethyl-ester was tested in the concentrations 25 nM to 20 mM, as described in Example 1, With HT22 cells, SK-N-MC cells, NIH3T3 cells and rat brain membranes. The result is shown in FIG. 2.

### EXAMPLE 4

N-dodecanoyl-tryptophan-ethyl-ester was tested in the concentrations 25 nM to 100 mM, as described in Example 1, with HT22 cells, SK-N-MC cells, NIH3T3 cells and rat brain membranes. The result is shown in FIG. 3.

1. Use of a D- or L-tryptophanyl-ester or its N-acyl derivative for producing a pharmaceutical composition for the prophylaxis and therapy of oxidative pathologic processes in degenerative diseases and/or cancer diseases.

2. Use according to claim 1, wherein the D- or L-tryptophanyl-ester or its N-acyl derivative is the following substance:
tryptophanoctyl-ester, tryptophandodecyl-ester, tryptophanstearyl-ester, tryptophanpalmityl-ester, tryptophanoleyl-ester, N-acetyl-tryptophanoctyl-ester, N-acetyl-tryptophandodecyl-ester, N-acetyl-tryptophanstearyl-ester, N-acetyl-tryptophanpalmityl-ester, N-acetyl-tryptophanoleyl-ester, N-dodecanoyl-tryptophanoctyl-ester, N-dodecanoyl-tryptophandodecyl-ester, N-dodecanoyl-tryptophanstearyl-ester, N-dodecanoyl-tryptophanpalmityl-ester, N-dodecanoyl-tryptophanoleyl-ester, N-acetyl-tryptophanethyl-ester, N-hexoyl-tryptophanethyl-ester, N-octoyl-tryptophanethyl-ester, N-dodecanoyl-tryptophanethyl-ester, N-stearoyl-tryptophanethyl-ester, N-palmitoyl-tryptophanethyl-ester or N-oleoyl-tryptophanethyl-ester.

3. Use according to claim 1, wherein the D- or L-tryptophanethyl-ester or its N-acetyl derivative is tryptophanoctyl-ester, N-oleoyl-tryptophanethyl-ester or N-dodecanoyl-tryptophanethyl-ester.

4. Use according to any one of claims 1 to 3 for the treatment and or prophylaxis of neurodegenerative diseases.

5. Use according to claim 4, wherein the neurodegenerative disease is Alzheimer's disease, Parkinson's disease, apoplectic fit or amyotrophic lateral sclerosis.

6. Use according to any one of claims 1 to 3 for the treatment and/or prophylaxis of cataracts.

7. Use according to claim 6, wherein the cataract is Cataracta senilis, Cataracta juvenilis, Cataracta diabetics or Cataracta complicata.

8. Use according to any one of claims 1 to 3 for the treatment and/or prophylaxis of neoplastic diseases.

9. Use according to claim 8, wherein the neoplastic disease is the prostatic carcinoma, the mammary carcinoma, the liver carcinoma, the kidney carcinoma, the colon carcinoma, the gastric carcinoma or the lung (bronchial) carcinoma.

10. Use according to any one of claims 1 to 3 for the treatment and/or prophylaxis of cardiovascular diseases.

11. Use according to claim 10, wherein the cardiovascular disease is arteriosclerosis or a myocardial infarction.

12. Pharmaceutical composition comprising a D- or L-tryptophanyl-ester or its N-acyl derivative according to any one of claims 1 to 3 for the prophylaxis and/or therapy of oxidative pathologic processes in degenerative and/or cancer diseases and, optionally, a pharmaceutically acceptable carrier, alone or in combination with other antioxidatively effective pharmaceutical compositions or pharmaceutical compositions used with degenerative and/or cancer diseases.

13. Use for the prophylaxis and/or therapy of oxidative pathologic processes in degenerative and/or cancer diseases comprising the administration of a prophylactic or therapeutic amount of a D- or L-tryptophanyl-ester or its N-acyl derivative according to any one of claims 1 to 3.
